(19) United States
(12) Patent Application Publication
(10) Pub. No.: US 2010/0210474 A1
(43) Pub. Date: Aug. 19, 2010

(54) HETEROCYCLIC SCAFFOLDS USEFUL FOR PREPARATION OF COMBINATORIAL LIBRARIES, LIBRARIES AND METHODS FOR PREPARATION THEREOF

(76) Inventor: Gary Gellerman, Rishon LeZion (IL)

Correspondence Address: The Law Office of Michael E. Kondoudis, 888 16th Street, N.W., Suite 800, Washington, DC, 20006, US

(21) Appl. No.: 12/678,300

(22) PCT Filed: Sep. 16, 2008
(86) PCT No.: PCT/IB2008/053758

§ 371 Date: Mar. 16, 2010

(30) Foreign Application Priority Data
Sep. 17, 2007 (IL) 186004

(51) Int. Cl. C40B 30/04; C40B 40/04; C40B 50/14; C07D 487/08; C07D 241/04; C07D 487/18

(52) U.S. Cl. 506/9; 506/15; 506/30; 540/460; 544/383

(57) ABSTRACT

Disclosed are heterocyclic scaffolds useful, for example, for solid-phase organic synthesis of combinatorial libraries and methods for the preparation thereof. Also disclosed are libraries, including combinatorial libraries, and methods for preparation thereof. Exemplified are the following scaffolds (I):

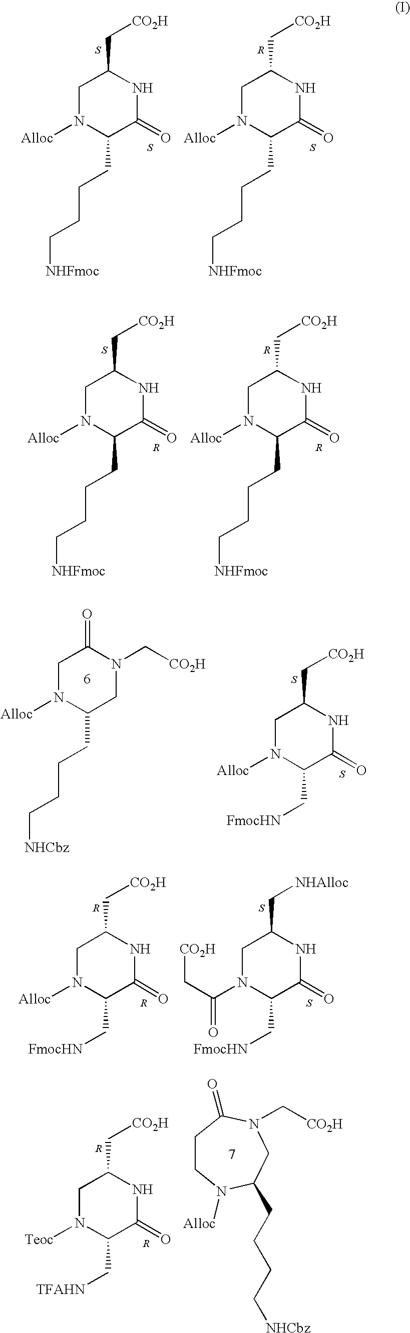

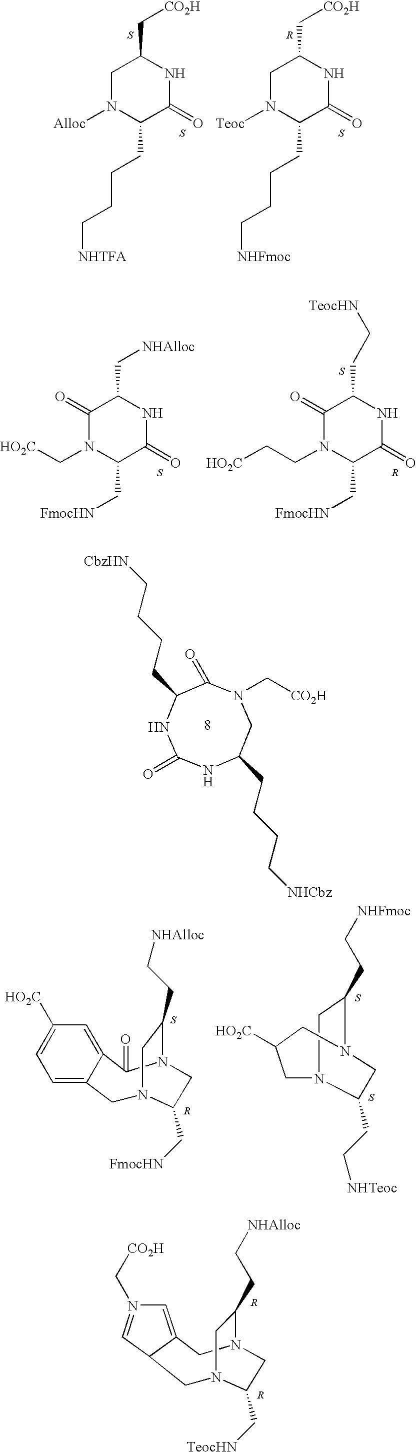

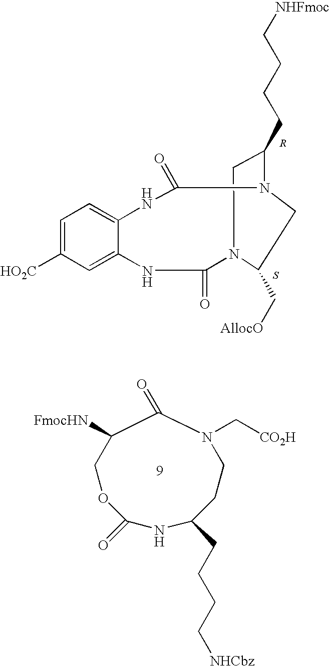

### RELATED PATENT APPLICATION

The present application gains priority from Israel patent IL 186,004 filed 17 Sep. 2007 which is included by reference as if fully set forth herein.

### FIELD AND BACKGROUND OF THE INVENTION

The invention, in some embodiments, relates to the field of drug-design and to the development of new drug lead compounds via combinatorial chemistry, while using solid-phase organic synthesis (SPOS). Specifically, some embodiments of the invention relate to heterocyclic scaffolds (e.g., have a piperazine, ketopiperazine, diketopiperazine, diazepane or pyrrolidone moieties) that in some embodiments are efficiently derivatizable and in some embodiments used to provide combinatorial libraries useful for drug design.

Parallel synthesis, and split and mix synthesis result with a large number of synthesized compounds, and the use of these techniques is an important tool in the search for new compounds in the pharmaceutical industry. Parallel synthesis is a particular form of chemical synthesis where a large number of chemical syntheses are performed separately to obtain a large number of new single discrete compounds. Split and mix synthesis is another form for organization of organic synthesis where a large number of compounds are synthesized as mixtures of compounds. Combinatorial chemistry is a form of parallel synthesis, and split and mix synthesis where the order and the features of the individual steps are performed using a particular combinatorial approach.

Combinatorial chemistry has recently emerged as an effective method for preparing large numbers of chemical compounds for use, e.g., in the discovery of biologically-active agents such as pharmaceutical drugs. In general, combinatorial chemistry is used to prepare compounds libraries in which all the members of the library share a common core structural element (a scaffold). Such libraries can be prepared by a variety of methods, including solution-phase synthesis and solid-phase organic synthesis.

Solid-phase organic synthesis alone or in combination with post-cleavage derivatization is a technology to perform parallel, and split and mix synthesis. In a solid-phase organic synthesis, a substrate (scaffold) is covalently linked to a suitable solid or insoluble support material, which can be a bead, a polymeric resin such as polystyrene or polystyrene copolymer polymer, through a linker, and after the solid-phase portion of the synthesis of compounds by derivatization of the scaffold is complete, the products are cleaved from the support material. In certain cases, solution phase synthesis steps are performed after cleavage from the support material to obtain the desired final products.

Combinatorial chemistry is an essential component of the drug discovery process. Using a split-mix synthesis procedure, chemical libraries can be generated so that each particle of support material (e.g., bead) displays only one compound entity, and an on-bead screening assay enables a rapid screening of a large number of compound-beads against specific molecular targets. Individual positive beads can then be isolated for the structure determination. This approach has been successfully applied to the identification of ligands for a large number of biological targets.

Structure determination of small molecule-beads with Edman degradation using an automatic protein sequencer is not easy. Various indirect encoding methods have been developed to sequence small molecule-beads more readily. Some methods synthesized a coding tag (comprising a coding building block and a coding linker) on each bead in addition to the library component, in order to define the chemical history of any particular bead and hence the structure of the compound it supports. The coding tag is released from the bead following biological screening and analyzed by a highly sensitive analytical technique.

Current chemical encoding methods have played an important role in the advancement of one-bead-one-compound combinatorial chemistry. However, those methods often require orthogonal chemistries for encoding, and therefore additional synthetic steps. In addition to the increased time and cost, the tagging molecules themselves potentially could interfere with the binding of the target entity (e.g., protein) to the library compounds bonded to the beads.

A recently developed peptide-based encoding method enabled practitioners to topologically segregate the testing compounds from the coding tags: resin beads are first derivatized with orthogonal protecting groups in the outer and inner regions separately. A coding tag precursor consisting of a sequence of α-amino acids, of which the side chains can be derivatized, is then constructed in the interior of the beads. During the library synthesis, building blocks are coupled to the outer scaffold and the side chains of the inner coding peptide simultaneously, so that the extra synthetic steps for coding the building blocks are eliminated by combining them with the library synthesis. After biological screening, the structures of active compounds is determined by direct sequencing of the coding peptides with Edman degradation. However, like other encoding strategies, this method has several limitations: a) it is based on Edman degradation, and therefore, is slow and expensive; b) building blocks have to be carefully chosen to avoid retention time overlap of their amino acid derivatives during sequencing; c) the choices for scaffolds are limited to those having the same functional groups as the side chains of commercially available trifunctional amino acids; and d) the removal of the final product from the reaction mixture by standard methods is difficult.

WO 2004/087933, incorporated by reference as if fully set-forth herein, describes the preparation of a library of compounds, using the derivatization of scaffold building blocks on solid support. WO 02/053546 describes the preparation of a library of compounds and scaffold building blocks, utilizing a solid support. EP 1 310 510 relates to molecular scaffolds synthesized on a solid support.

According to Thorpe [Thorpe, D. S., The Pharmacogenomics. J. 1 (2001) 229-33], the piperazine template is defined as a “privileged scaffold”—a molecular backbone with versatile binding properties representing a frequently-occurring binding motif, and providing potent and selective ligands for a range of different biological targets. The high number of positive hits revealed in biological screens with the piperazine template urged chemists to develop plenty of different synthetic methods that allow for the fast and efficient building of these heterocyclic system. Some of the previously published methods enable assembly of the piperazine scaffold on solid support while some describe the synthesis by solution chemistry. One of the most fascinating methods, relates to multicomponent reactions, seem to be particularly well suited to assemble piperazines introducing extremely high diversity “around the scaffold” [Doemling A., “Convergent and Fast Route to piperazines via IMCR” Org. Chem. Highlights, 2005]. However, in most cases, this methodology is not enantiospecific related to the carbons in piperazine template, generating mixtures of stereoisomers, and necessitating thorny purification.

It would be useful to have scaffolds that are suitable for use in drug-design for the efficient synthesis of libraries of chemical compounds having a piperazine and related structures.

### SUMMARY OF THE INVENTION

Some embodiments of the present invention provide heterocyclic scaffolds (e.g., have a piperazine, ketopiperazine, diketopiperazine, diazepane or pyrrolidone moiety) chemical compounds that are useful scaffolds for synthesis, for example in the field of drug-design and combinatorial chemistry, for example for the synthesis of drug candidates or for the preparation of combinatorial libraries.

In some embodiments, the scaffolds have three orthogonally-protected groups. In some embodiments, the scaffolds are synthesized using solution-phase chemistry, which in some embodiments, allows the production of large amounts of scaffolds of high purity, including optical purity.

In some embodiments, the scaffold are attached to a solid-phase organic synthesis (SPOS) support material (e.g., a bead or resin) and subsequently the two remaining orthogonally protected groups are derivatized using SPOS.

In some embodiments, the teachings of the present invention provide combinatorial libraries comprising a plurality of chemical compounds having a heterocyclic scaffold as described herein.

Thus, unlike the art where pharmaceutically-significant libraries of small-molecule are tedious to make, some embodiments the present invention allows the simple preparation of heterocyclic scaffolds of high purity, that can be derivatized, for example for preparing combinatorial libraries, with few steps.

Some embodiments allow preparation of chiral orthogonally-protected heterocyclic scaffolds in solution followed by attaching the scaffolds to SPOS supports for derivatization using SPOS.

Therefore, it is an object of some embodiments of the invention to provide substantially optically-pure molecular scaffolds synthesized in-solution. It is an object of some embodiments of the invention to provide a library of compounds, for example for drug discovery, comprising heterocyclic compounds synthesized on a heterocyclic scaffold using SPOS. It is an object of some embodiments of the invention to provide a library comprising chiral scaffolds.

According to an aspect of some embodiments of the invention is provided an orthogonally-protected, heterocyclic, chiral scaffold having a structure selected from the group consisting of formulae (I) or (II):

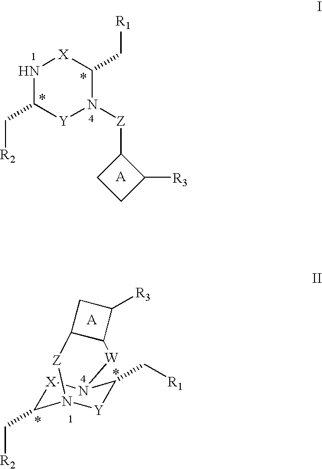

wherein:
* indicates chiral centers;
X, Y, Z and W are independently selected from CH₂ and C═O; R₁ is selected from —(CH₂)ₖ—COOH and —(CH₂)ₖ-Q1-P¹; R₂ is selected from —(CH₂)ₘ—COOH and —(CH₂)ₘ-Q2-P²; R₃ is selected from —(CO)ₚ-A-COON and —(CO)ₚ-A-Q3-P³; p=0 or 1 Q1, Q2, and Q3 are linkers independently selected from N, NH, O, and S; A is a linker selected from —(CH₂)ₙ, —O—(CH₂)ₙ, —NH—(CH₂)ₙ, —N-Alkyl-(CH₂)ₙ, phenylene-(CH₂)ₙ, -cyclopropylene-(CH₂)ₙ, -cyclobutylene-(CH₂)ₙ, -cyclopentylene-(CH₂)ₙ, -cyclohexylene-(CH₂)ₙ, -piperidinylene-(CH₂)ₙ, pyrrolene-(CH₂)ₙ; P¹, P², and P³ are groups protecting the adjacent respective linkers Q1, Q2, and Q3; and wherein k, m, and n are integers selected from 0 to 5.

In some embodiments, the scaffolds are small having a molecular weight of less than about 350 in the deprotected form, e.g., when the protecting groups P₁, P₂ and P₃ are replaced with H or OH, as relevant.

In some embodiments, the scaffold is substantially optically-pure and comprises a substantially pure isomer selected from the group consisting of RR, RS, SR, and SS, wherein R and S describe the isomeric configuration at the two chiral centers of the scaffold.

In some embodiments, the protecting groups P¹, P², and P³ are orthogonal, that is may be removed under different conditions. The protecting groups are any suitable protecting groups. In some embodiments, the protecting groups P¹, P², and P³ are independently selected from the group consisting of Alloc, Fmoc, TPA, CBZ, Boc, o-Nosyl, Mtt, Ddz Dde, Bpoc, NVOC, NBoc and Teoc if the adjacent linker is NH, from the group consisting of Alloc, Allyl, Bz, Dmb, Fmoc, Pivaloyl and Ac if the adjacent linker is O, and from the group consisting of Acm, Trt and StBu if the adjacent linker is S.

According to an aspect of some embodiments of the invention is provided a method of synthesizing a compounds comprising: a) providing a scaffold of Formula (I) or (II); b) attaching the scaffold to a support useful in SPOS through one of R₁, R₂ and R₃; and, c) subsequently to ‘b’, using SPOS to derivative the scaffold by serially deprotecting and derivatizing at least one of R₁, R₂ and R₃.

According to an aspect of some embodiments of the invention is provided an orthogonally-protected, heterocyclic, chiral scaffold having a structure selected from the group consisting of formulae (I) or (II), for use in SPOS.

According to an aspect of some embodiments of the invention is provided a library of compounds comprising at least one orthogonally-protected, heterocyclic, chiral scaffold having a structure selected from the group consisting of formulae (I) or (II). According to an aspect of some embodiments of the invention there is provided a library of compounds, comprising at least one compound including a scaffold as described herein. According to some embodiments, the library comprises at least two different compounds including the same scaffold. According to some embodiments, the library is non-combinatorial. According to some embodiments, the library of compounds is combinatorial and comprises a plurality of different combinatorially-varying compounds including the same scaffold. According to some embodiments, the library comprises a collection of a plurality of different compounds of formula (I) and/or (II), variably derivatized at R₁ and/or R₂ and/or R₃. It is important to note that a person skilled in the art provided with the scaffolds described herein is able to prepare and use a library of the invention without undue experimentation using methods known in the art and adequately described in the literature.

According to an aspect of some embodiments of the invention is provided for the use of said library in identifying a pharmaceutically important ligand. More specifically, said library comprises a moiety exhibiting high affinity toward a receptor present on a targeted cell.

According to an aspect of some embodiments of the invention there is provided a method of identifying a compound having a pharmaceutically significant interaction with a biological target, comprising: a) providing a compound comprising a scaffold as described herein; b) contacting a biological target with the compound; and c) determining if the affinity between the compound and the biological target is pharmaceutically significant. It is important to note that a person skilled in the art provided with the scaffolds described herein is able to implement the method of identifying a compound without undue experimentation using methods known in the art and adequately described in the literature.

According to an aspect of some embodiments of the invention is provided a combinatorial preparation of a pharmaceutically active heterocyclic compound, having a high affinity toward a pharmaceutically relevant receptor, comprising:

i) preparing an orthogonally-protected, heterocyclic, chiral scaffold in-solution having a structure selected from the group consisting of formulae (I) or (II) (the symbols have the same meaning as above):

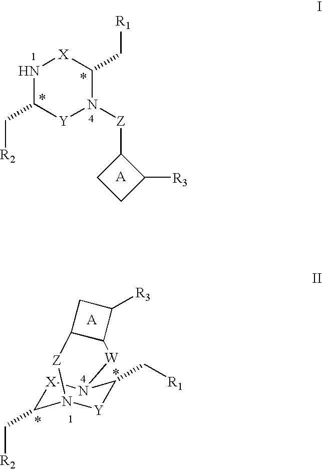

ii) derivatizing said chiral scaffold (for example, at R₁, R₂ and/or R₃) to obtain a desired biologically active precursor lead-compound, either in-solution or attached to a solid support (e.g., a bead or resin, such as known in the field of SPOS);

iii) contacting the resulting chiral lead compound with said receptor (biological target molecules);

iv) releasing said chiral biologically active compound and evaluating its affinity toward biological targets (e.g. receptors); and

v) further optimizing the leads from these libraries by using the same or other from the pool set of scaffolds.

According to an aspect of some embodiments of the invention there is provided a method of identifying a compound having a pharmaceutically significant interaction with a biological target, comprising: a) providing a library as described herein; b) contacting a biological target with compounds making up the library; and c) determining if the affinity between the compounds of the library and the biological target is pharmaceutically significant. It is important to note that a person skilled in the art provided with the scaffolds described herein is able to implement the method of identifying a compound without undue experimentation using methods known in the art and adequately described in the literature.

According to an aspect of some embodiments of the invention there is provided a method for solution synthesis of a substantially optically-pure heterocyclic scaffold from chiral amino acid synthons, comprising: protection and deprotection steps of a compound of formula (I) or (II), and purification.

According to an aspect of some embodiments of the invention there is provided a method of preparing a scaffold as described herein, comprising providing lysine or a derivative thereof, N-protected at both amino groups; and reducing the lysine with a hydride.

According to an aspect of some embodiments of the invention there is provided a method of synthesizing a library of chiral heterocyclic compounds, comprising i)

providing heterocyclic scaffolds as described herein; ii) attaching the scaffolds to supports useful for solid-phase organic synthesis; iii) preparing a plurality of different compounds by variably substituting the scaffolds attached to the supports; wherein the plurality of different compounds constitute the library. According to some embodiments, the compounds attached to the supports constitute the library. According to some embodiments, the method further comprises iv. releasing the compounds form the supports, whereby the compounds released from the supports constitute the library. It is important to note that a person skilled in the art provided with the scaffolds described herein is able to implement the method of synthesizing a library of compounds based thereupon without undue experimentation using methods known in the art and adequately described in the literature.

Unless otherwise defined, technical and/or scientific terms used herein have the same meaning as commonly understood by one of ordinary skill in the art to which the invention pertains. Examples include terms and abbreviations for groups and chemicals used in solid-phase chemical synthesis including: Alloc: allyloxycarbonyl; Fmoc: 9-fluorenylmethyl chloroformate; Boc: tert-Butyloxycarbonyl; Teoc: 2-(Trimethylsilypethoxycarbonyl; TFA: trifluoroacetamide; Ac: Acetyl; CBZ: Carboxybenzyl; Acm: acetamidomethyl; DIC: diisopropylcarbodiimide; StBu: tert.-butylmercapto; DCC: Dicyclohexylcarbodiimide; HOBt: 1-hydroxybenzotriazole hydrate; Dmb: dimethoxybenzyl; Ddz: dimethoxy dimethyl benzyloxycarbonyl; Mtt: methyl trityl; o-Nosyl: 4-nitrobenzenesulfonyl; Dde: 2-acetyl dimedone; Bpoc: 2-(p-biphenylyl)-2-propyloxycarbonyl; NVOC: 6-Nitroveratryloxycarbonyl; NBoc: n-butoxycarbonyl; TBTU: O-(Benzotriazol-1-yl)-N,N,N′,N′-tetramethyluronium tetrafluoroborate; PyBoP: benzotriazol-1-yl-oxytripyrrolidinophosphonium hexafluorophosphate; HATU:2-(7-azabenzotriazol-1-yl)-1,1,3,3-tetramethyluronium hexafluorophosphate; PyBroP:bromotripyrrolidinophosphonium hexafluorophosphate; EtAc: ethylacetate; DCM: dichloromethane; PE: petroleum ether; MeOH: methanol; NMM: N-methylmorpholine; DMSO: dimethylsulfoxide; DMF: dimethylformamide; AcOH: acetic acid; THF: tetrahydrofuran; and DIEA: N,N-Diisopropylethylamine.

As used herein, the term scaffold (in some instances referred to as substrate or building block) is used as known in the field of combinatorial chemistry, that is a derivatizable chemical structure that serves as a common core structural element of a group of chemicals, for example chemicals making up a combinatorial library.

The materials, methods, and examples disclosed herein are illustrative only and are not intended to be necessarily limiting.

As used herein, the terms “comprising”, “including” and “having” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, the indefinite articles “a” and “an” mean “at least one” or “one or more” unless the context clearly dictates otherwise.

### BRIEF DESCRIPTION OF THE DRAWINGS

The above and other characteristics and advantages of the invention will be more readily apparent through the following examples, and with reference to the appended drawings, wherein:

FIG. 1 depict an embodiment of the preparation of a diketopiperazine (DKP) scaffold;

FIGS. 2A-2G together depict an embodiment of the preparation of a ketopiperazine scaffold;

FIG. 3 depicts general structures of embodiments of orthogonally protected substantially optically-pure keto- and diketopiperazine, 2-ketodiazepane and 3-aminopyrrolidone scaffolds;

FIG. 4 depicts an embodiment of the preparation of substantially optically-pure Alloc/Fmoc orthogonally protected ketopiperazine 1;

FIG. 5 depicts an embodiment of the preparation of substantially optically-pure Alloc/Fmoc orthogonally protected diketopiperazine 2;

FIG. 6 depicts an embodiment of the preparation of Alloc/Boc and Alloc/Fmoc orthogonally protected ketopiperazines 3 and ketodiazepane 4; and

FIG. 7 depicts an embodiment of the preparation of Cbz/Boc and CBz/Fmoc orthogonally protected aminopyrrolidone 5.

### DESCRIPTION OF SOME EMBODIMENTS OF THE INVENTION

It has now been found that a library of heterocyclic lead compounds can be synthesized using in-solvent synthesis of heterocyclic chiral protected scaffolds, followed by their derivatization when attached to a solid support (e.g., beads or resins known in SPOS). In accordance with some embodiments of the invention, a collection of chiral, orthogonally-protected heterocyclic scaffolds are prepared free in the solution, ready for diversification by SPOS (solid-phase organic synthesis) for generating libraries on solid support. In order to preserve important chiral centers, some embodiments of the invention provide the use of piperazine templates initially prepared in substantially optically-pure form that bear various tethers with orthogonally-protected groups applicable in SPOS. Some embodiments of the invention describe the synthesis of the heterocyclic scaffolds of the formula I and II that are applied in “around-the-scaffold” diversification by SPOS, exhaustively sampling the medicinally relevant space, introducing valuable physico-chemical properties in three independent directions.

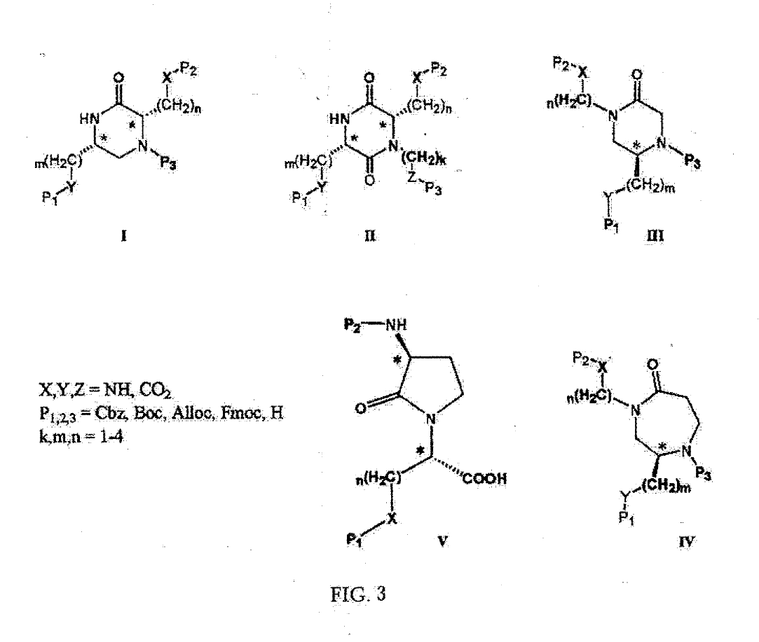

Typical heterocyclic scaffolds are depicted herein and in the Figures, see for example FIG. 3.

The basis of some embodiments of the invention is the preparation of a sufficient pool of orthogonally-protected heterocyclic scaffolds for generation of multifunctional piperazine and keto analog libraries. Once the piperazine motif is chosen for optimization, the pre-designed small library from a few scaffolds around a desired motif is prepared and screened. The essential information for further optimization of drug-like properties of obtained hits is optionally performed around the same or other from the pool of scaffolds. This is a novel operation, enabling to manage fast and efficient multi-cyclic optimization process around heterocyclic template. The molecules from these libraries have already piperazine template and may further be optimized by using the same or other scaffolds from the pool of scaffolds.

The novel compounds of formulae (I) to (II) of the invention, also called orthogonally-protected scaffolds, are prepared in some embodiments as free soluble molecules, not bound to any solid support using solution chemistry techniques. In some embodiments, said scaffolds are used as precursors for the synthesis of a library of complex molecules, that are useful, for example, as lead compounds in drug development.

In some embodiments, the invention further teaches the preparation of substantially optically-pure, orthogonally-protected heterocyclic scaffolds utilization in the generation of substantially optically-pure libraries. The use of said substantially optically-pure scaffolds, enables the production of substantially optically-pure library members, which can then be biologically screened. These and more will be detailed below.

In general, some embodiments of the present invention involve providing in-solution orthogonally-protected heterocyclic scaffolds and their attachment to a solid-supported linker group such as known in the art of SPOS. Such attached scaffolds can then be modified by stepwise reaction under a selected reaction scheme (using SPOS) until a desired product is obtained. The desired compound can then be cleaved from the solid support under mild conditions which do not significantly destroy or modify the desired compound.

Some embodiments of the invention are performed under a wide range of conditions, though it will be understood that the solvents and temperature ranges recited herein are not limiting and only correspond to specific embodiments of the invention. A variety of synthetic methods are compatible with some embodiments of the derivatization of the invention, e.g. amidation, nucleophilic substitution, cycloadditions, aldol reactions, and the like. In general, it is desirable that reactions are run using mild conditions that will not adversely affect the substrates, the intermediates, or the products. In certain embodiments it is preferable to perform the reactions under an inert atmosphere of a gas such as nitrogen or argon.

Some embodiments of methods of the invention are described in the examples below for synthesizing the scaffolds and the compounds are advantageous over the known methods in several ways, including a) yielding substantially optically-pure products, and b) utilizing both solid-phase and in-solution synthesis. Novel orthogonally-protected substantially optically-pure piperazine, ketopiperazine and diketopiperazine scaffolds of the invention are advantageous for generating libraries by SPOS, and in optimizing synthesis “around the scaffold”. Some embodiments of the invention overcome the above-mentioned limitations, providing novel approaches for improving the production process, the rate, better product selectivity, lower toxicity and higher purity of the products.

Some embodiments of the novel heterocyclic scaffolds of the invention are versatile, constrained, and medicinally relevant chiral templates, functionalized with three anchors for independent 3D evolution of the drug-like properties in core structures. soluble (ClogP 1-4, where ClogP, the lipophilicity coefficient, should be below 4 for pharmaceutically acceptable bioavailability) and are considered to be a good source for lead compounds. Some embodiments of the scaffolds are suitable for some known state of-the-art lead optimization method (e.g. HT Crystallography, SAR by NMR, etc.). The use of such scaffolds may allow for the efficient optimization of the Medicinally valuable features of a drug (e.g. selectivity, affinity, toxicity, oral bioavailability). Some such scaffolds retain their chiral properties and optical purity to the final lead compounds of the libraries; that will be biologically screened.

Some embodiments of the invention relate to Chiral piperazines (in some cases substantially optically-pure) with a plurality of orthogonally-protected sites ready for SPOS which are selected from the group consisting of;

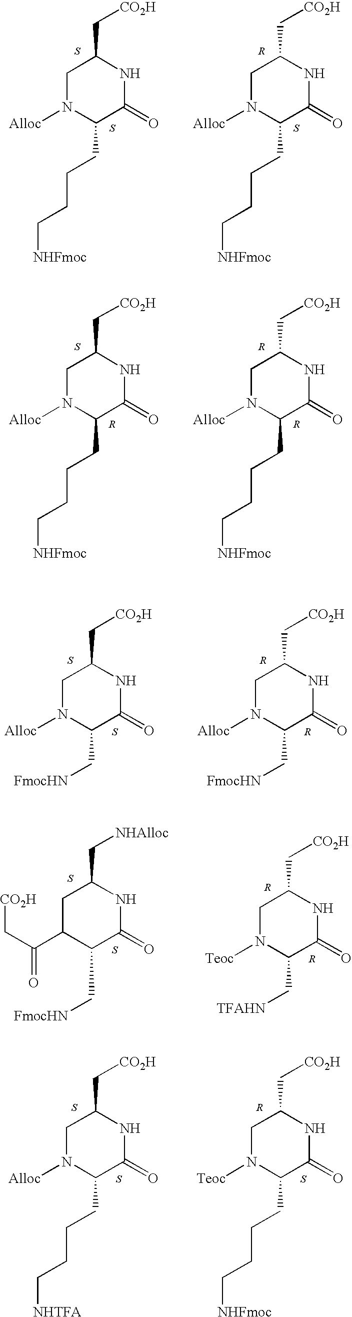

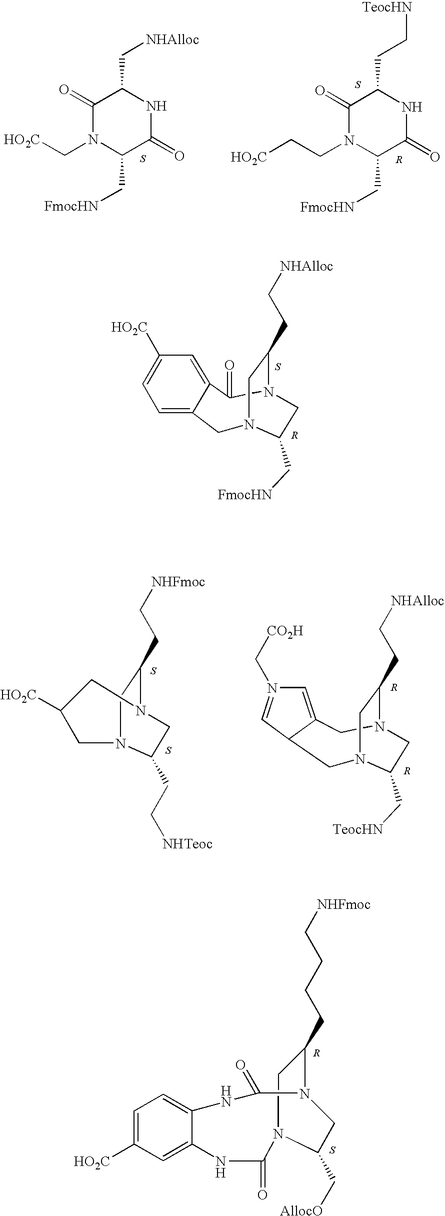

Another embodiment of the invention relates to the production of ketopiperazine scaffolds, and keto- and diketo-diazacyclic compounds of the formula:

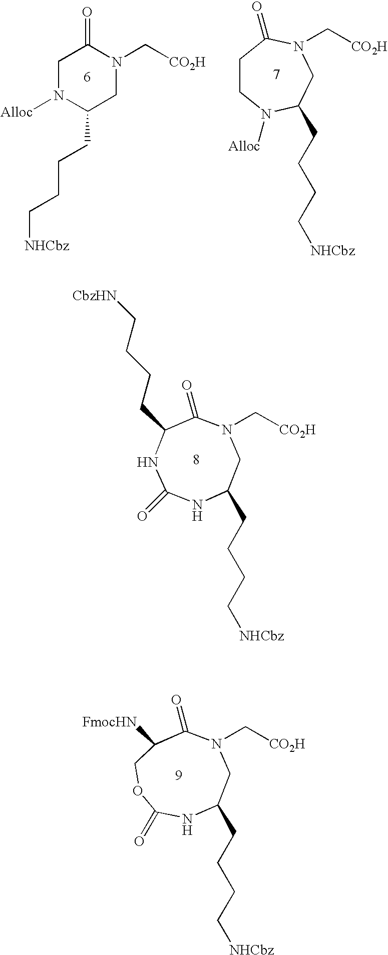

An aspect of some embodiments of the invention is a combinatorial method of preparing a pharmaceutically-active heterocyclic compounds and possibly based on other diaza cyclic compounds, having a high affinity toward a pharmaceutically relevant receptor, comprising a) binding of an orthogonally-protected, heterocyclic, chiral scaffold to a solid surface; and b) reacting said scaffold with a suitable reactant or synthon thereby obtaining chiral lead compound; and c) releasing said lead compound and evaluating its affinity toward said receptor.

In some embodiments, a scaffold according to the invention has a structure selected from:

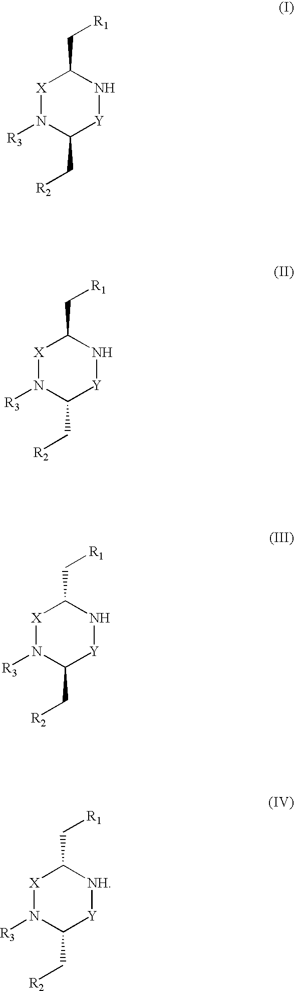

wherein X and Y are independently selected from CH₂ and C═O; R₁ is selected from —COOH, —NH-Alloc, —NH-Teoc; R₂ is selected from —(CH₂)ₙ—NH-Fmoc, —(CH₂)ₙ—NH-TFA; and R₃ is CBZ.

Some embodiments of the heterocyclic scaffolds of the invention may be synthesized by established solution synthesis methods; some embodiments of the scaffolds are advantageously assembled from chiral amino acid synthons, preserving the chirality of the chiral center in the scaffolds. Said methods may comprise reductive amination with NaBH₃CN or NaBH(OAc)₃; amide bond formation using DIC, DCC/HOBt, TBTU, PyBoP, HATU, PyBroP; protection, deprotection of Boc (trifluoroacetic acid/DCM), Fmoc (Piperidine), Teoc (TBAF), Alloc (Pd/Tetrakis), CBZ (H₂, Pd(OH)₂), cyclization (heat, Toluene/2-butanol), etc. The final protected scaffolds (precursors) are purified by flash chromatography (EtAc/DCM/Hexane, PE, MeOH) in multi gram scale. Diversification and elongation of tethers is achieved by SPOS (e.g. amination, alkylation, amidation, acylation, esterification). The release from the resin can be done for acid sensitive resins with 95% trifluoroacetic acid/2.5% H₂O/2.5% Tris; for super acid sensitive resins with 1% trifluoroacetic acid/DCM; or by reductive mode (NaBH₄) for releasing from the resin in OH form. The final members of the library are purified by HPLC in 2-10 mg scale.

Some embodiments of the invention further describe the synthesis of substantially optically-pure, orthogonally-protected heterocyclic scaffolds of the general structures (I) and (II) that are applied in “around-the-scaffold” diversification by SPOS, introducing valuable physico-chemical properties in three independent directions. This enables the preparation of sufficient pool of orthogonally-protected substantially optically-pure piperazine based scaffolds for fast generation of substantially optically-pure multifunctional piperazine and its keto/aza analog libraries. These medicinally relevant scaffolds are small, substantially optically-pure, relatively constrained and bear three arms with different functional groups such as amine, carboxyl, hydroxyl and thiol in various combinations. In the scaffolds, all or some of the functional groups are orthogonally-protected, in some embodiments there is an unprotected carboxyl which is used for loading of the scaffold on resin or other SPOS support. The protecting groups applicable in SPOS may include: Alloc, Fmoc, Teoc, TFA for amines; Alloc, Allyl, Fmoc, Pivaloyl, Acetyl for hydroxyls; Acm and StBu for thiols. The spacers incorporated in the ketopiperazine scaffolds optionally introduce additional level of diversification. In some embodiments, once the piperazine motif is chosen for optimization, the pre-designed small library from a few scaffolds around desired motif is prepared by SPOS and screened. Further optimization of the drug-like properties of the acquired hits is optionally performed around the same scaffold or around other scaffolds from the pool of scaffolds. Such an operation enables to manage a fast and efficient, novel, multi-cyclic optimization process around heterocyclic scaffold. Furthermore, being chiral and controllable in length and nature of the side arms, some embodiments of the scaffolds yield heterocyclic libraries with high-resolution coverage of medicinal space around chosen heterocyclic motif. The molecules from these libraries have already heterocyclic template and can be further optimized rapidly by using the same, or other from the pool of scaffolds.

These and other aspects of the invention will become clear from the following examples, which are illustrative only and do not limit the invention.

### EXAMPLES

### General

Analytical HPLC was performed on a 250×4.2 mm Lichroprep RP-18 column from Merck (Whitehouse Station, N.J., USA), with a 1 ml/min flow and detection at 214 nm. The eluents were triply distilled water and HPLC-grade CH₃CN (containing 0.1% trifluoroacetic acid) or MeOH. Optical rotations were recorded at 25° C. in a 10 cm length cell and [a]_{D}-values are given in units of 10⁻¹ deg cm²/g. The concentration of all the samples was 0.5%. Mass spectra were measured in the positive and negative modes using a quadrupole mass spectrometer equipped with an electrospray ionization source and cross-flow inlet. ¹H and ¹³C NMR spectra were recorded at 300 and 75 MHz, respectively in CDCl₃, unless otherwise indicated. Assignments in the final products were supported by 2D COSY, TOCSY, NOESY, ROESY, HMBC and HMQC spectroscopy. All chemical shifts are reported with respect to TMS. Chromatography was carried out by standard flash chromatography and TLC on silica-gel (Merck 7735).

Unless otherwise stated, chemicals and materials were obtained from Sigma-Aldrich (St. Louis, Mo., USA).

### Example I

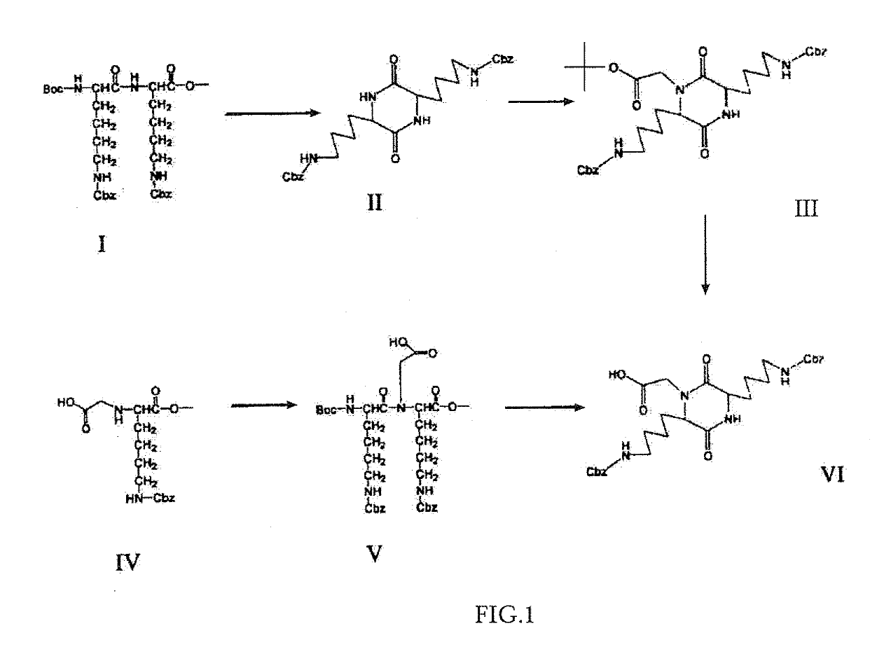

### Synthesis of a Diketopiperazine Scaffold (FIG. 1)

A solution of Boc-Lys(Cbz)-OH (12 gram, 0.0318 mol) [compound I in FIG. 1] in 70 ml ethyl acetate was cooled to −15° C., and treated with 7.67 ml (0.06996 mol) N-methylmorpholine (NMM), and then 5 ml (0.03816 mot) isobutyl chloroformate. 5 min later, Lys(Cbz)-OMe (10 gr, 0.0318 mol) was added and stirring was continued for 15 min at −15° C., and at room temperature for 45 min. The mixture was evaporated in vacuum, the residue taken up in 160 ml ethyl acetate and 120 ml water and the organic layer washed with cold water, 10% solution of citric acid in water, 0.5N potassium hydrogen carbonate, and twice in water and dried over anhydrous Mg₂SO₄. The solvent was removed in vacuum yielding 16.8 g of the product [compound II in FIG. 1]. MS (H⁺): 657.4, ¹H NMR (300 MHz, CDCl₃): δ=7.4 (m, 10H), 4.9 (s, 4H), 4.2 (m, 2H), 3.5 (s, 3H), 2.9 (m, 4H), 2.2 (m, 4H), 1.9-1.5 (m, 8H), 1.4 (s, 9H), 1.1 (m, 4H). MS=656.

Boc-dipeptide ester (compound I in FIG. 1) (16.62 gr, 0.0253 mol) was treated with 50 ml 4N HCl-dioxane at room temperature for 30 min, followed by the removal of excess HCl by repeated evaporation with dioxane in a vacuum (repeated three times). The resulting hydrochloride was dissolved in 0.1M AcOH (acetic acid)-2-butanol (250 ml), and NMM (2.77 ml, 0.253 mol) was added. The resulting weakly acidic solution was refluxed in an oil bath overnight. The product was collected on a filter, washed with small amounts of cold 2-butanol affording 10.3 g of diketopiperazine [compound III in FIG. 1]. MS (H⁺): 525.4; ¹H NMR (300 MHz, DMSO): δ=7.6-7.3 (m, 10H), 6.3 (s, 2H), 5.2 (s, 4H), 4.4 (t, 2H), 3.1 (m, 4H), 1.9 (m, 4H), 1.5-1.2 (m, 8H).

In a two-necked flask under nitrogen, compound II (1 gr, 0.0019 mol), BrCH₂COOt-Butyl (tBu) (0.28 ml, 0.0019 mol), Ag₂O (0.88 gr, 0.0038 mol), and DMF (15 ml) were mixed with stirring at room temperature. The resulting mixture was then heated at 40° C. for 48 h in an oil bath. It was subsequently filtered through a celite pad and the filtrate was concentrated to dryness under reduced pressure to afford crude compound III. The removal of t-Bu was done in trifluoroacetic acid/DCM 1:1 mixture at RT. After the evaporation of the solvent, the residue was purified by chromatography on a column with 3% MeOH in EtAc to give compound VI (FIG. 1). ¹H NMR (300 MHz, DMSO): δ=7.5-7.3 (m, 10H), 5.2 (s, 4H), 4.7 (m, 2H), 4.0 (s, 2H), 2.6 (m, 4H), 1.9 (m, 2H), 1.7 (m, 4H), 1.4 (s, 9H), 1.1 (m, 4H).

Lys(CBz)-OMe (1 g, 0.00315 mol) was added to 20 ml of MeOH/AcOH (99:1) and the solid was dissolved. 2-oxoacetic acid (0.35 gr, 0.00315 mol) was added in 5 ml of MeOH/AcOH (99:1) to the amino acid solution and the reaction mixture was stirred for 1 h. After stirring, NaCNBH₃ (0.27 gr, 0.004 mol) was added carefully. After 2.5 h, solvent was evaporated to give 0.92 gr of crude material [compound V in FIG. 1]. MS (H⁺): 353.2, ¹H NMR (300 MHz, DMSO) δ=7.9 (m, 5H), 5.1 (s, 2H), 3.6 (s, 3H), 3.4 (m, 3H), 2.9 (m, 2H), 1.7 (m, 2H), 1.5-1.2 (m, 4H).

To a solution of free amine (compound IV in FIG. 1) (4.9 gr, 0.013 mol) in 100 ml DMF were added. 4.58 gr (0.013 mol) of Boc(Z)-Lys-OH, HOBt 1.89 gr (0.014 mol) and diisopropylcarbodiimide (DIC) 2.2 ml (0.014 mol). The resulting mixture was stirred at room temperature overnight, and then DMF was evaporated under vacuum. The residue was dissolved in 100 ml of ethyl acetate, washed with aqueous NaHCO₃ and then dried over Mg₂SO₄. Purification by column chromatography (petroleum ether/ethyl acetate) gave 5.2 gr pure dipeptide [compound VI in FIG. 1]. ¹H NMR (300 MHz, DMSO): δ=7.6 (m, 10H), 5.0 (s, 4H), 4.7 (m, 2H), 4.0 (s, 2H), 3.8 (s, 3H), 3.1 (m, 4H), 1.8-1.4 (m, 8H), 1.4 (s, 9H), 1.1 (m, 4H).

Boc-CBZ-dipeptide ester (2.3 gr, 0.003 mol) was treated with 10 ml 4N HCl-dioxane at room temperature for 30 min, followed by removal of excess HCl by repeated evaporation with dioxane in a vacuum (repeated three times). The resulting hydrochloride was dissolved in 0.1M AcOH 2-butanol (30 ml) and NMM (0.3 ml, 0.003 mol) was added. The resulting weakly acidic solution was refluxed in an oil bath overnight. The product was collected on a filter, washed with small amounts of cold 2-butanol to yield 1.4 gr of pure diketopiperazine (compound VI in FIG. 1). MS (H⁺): 583.4; ¹H NMR (300 MHz, DMSO): δ=7.6-7.3 (m, 10H), 4.8 (s, 2H), 4.5 (m, 2H), 4.1 (s, 2H), 3.0 (m, 4H), 1.7 (m, 4H), 1.4-1.1 (m, 8H).

### Example 2

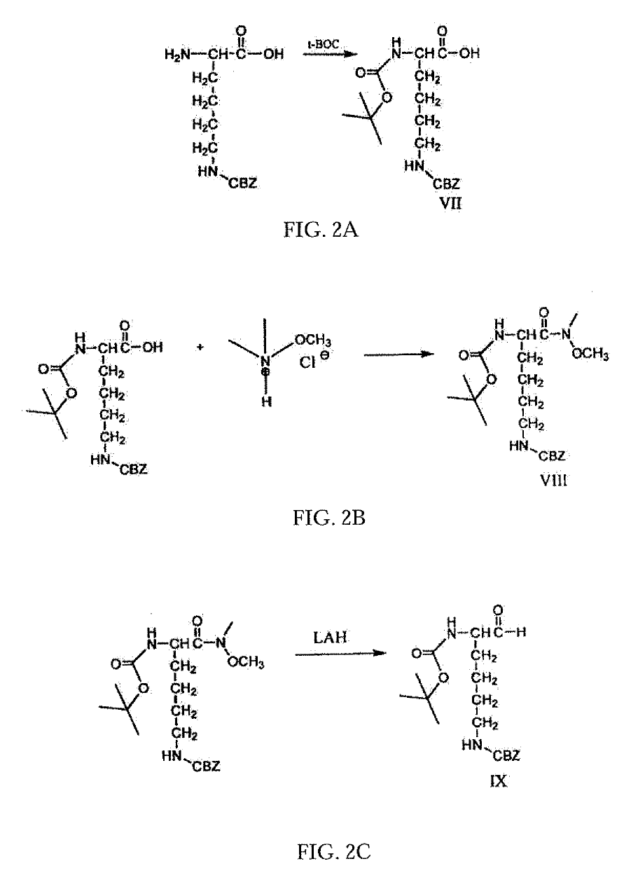

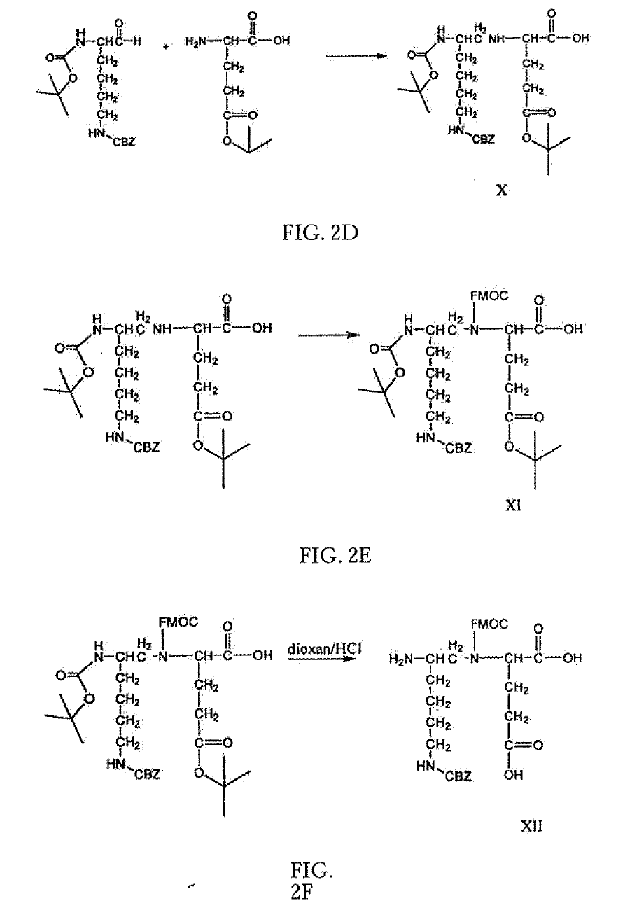

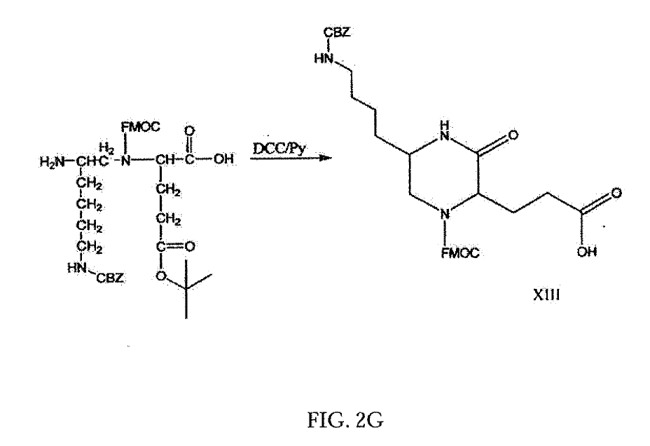

### Preparation of Ketopiperazine Scaffold (FIG. 2)

### FIG. 2A

### BOC-LYS-Z-OH

A solution of the amino acid (0.02 mol) in mixture of dioxane (40 ml), water (20 ml) and 1N NaOH (20 ml) was stirred and cooled in an ice water bath. Di-tert-butyl pyrocarbonate (0.022 mol) was added and stirring was continued at room temperature for 0.5 h. The solution was concentrated in vacuum to about 10 to 15 ml, cooled in an ice water bath, covered with a layer of ethyl acetate (60 ml) and acidified with a dilute solution of KHSO₄ to pH 2-3. The aqueous phase was extracted with ethyl acetate (30 ml) and the extraction repeated. The ethyl acetate extracts were pooled, washed with water (twice) dried over anhydrous Na₂SO₄ and evaporated in vacuum.

### FIG. 2B

### Hydroxamate

To a solution of Boc-Lys-Z-OH (0.012 mol) [compound VII in FIG. 2A] in DCM (40 ml) was added N-methylmorpholine (0.024 mol). The mixture was cooled to −15° C., and isobutylchlorofounate (0.012 mol) was added. The mixture was cooled to −15° C. for 15 min followed by the addition of N,O-dimethylhydroxyiamine hydrochloride (0.014 mol). The mixture was stirred at −15° C. for 1 h, allowed to warm to room temperature, and stirred for 3 h. The reaction mixture was poured into H₂O (40 ml), and the aqueous phase was extracted with DCM (2×40 ml). The combined organic extracts were dried over Na₂SO₄ and the solvent was removed in vacuum to give (96%) of clear oil. MS(H⁺): 424.3; ¹H NMR (300 MHz, CDCl₃): δ=7.3 (m, 5H), 5.1 (s, 2H), 4.2 (m, 2H), 3.6 (s, 3H), 2.7 (s, 3H), 2.2 (m, 2H), 1.9-1.5 (m, 4H), 1.4 (s, 9H), 1.1 (m, 2H).

### FIG. 2C

### Aldehyde

A mixture of hydroxamate (0.007 mol) [compound VIII in FIG. 2B], THF (100 ml) and LiAlH₄ (0.014 mol) was stirred under N₂ for 40 min in an ice bath. A solution of KHSO₄ (1.35 g) in 30 ml H₂O was added, and THF is removed under reduced pressure. The residue was extracted with ether (4×40 ml). The combined organic layers were washed with aqueous HCl (3×40 ml), sat. aq. NaHCO₃ (40 ml) and brine (40 ml) and dried (MgSO₄). Evaporation of the solvent in a rotary evaporator gave an aldehyde as a colorless oil, yield (80%). MS (H⁺): 365.3; ¹H NMR (300 MHz, CDCl₃): δ=9.8 (bs, 1H), 7.3 (m, 5H), 5.1, (s, 2H), 4.2 (m, 2H), 2.2 (m, 2H), 1.9-1.5 (m 4H), 1.4 (s, 9H), 1.1 (m, 2H).

### FIG. 2D

### Reductive Alkylation

The amino acid (0.005 mol) was added to 30 ml of MeOH/AcOH (99:1) until the solid was dissolved. Corresponding aldehyde (0.005 mol) [compound IX in FIG. 2] was added in 10 ml of MeOH/AcOH (99:1) to the amino acid solution and reaction mixture was stirring for 1 h. After stirring NaCNBH₃ (0.0065 mol) was added dropwise. The reaction was controlled by TLC. When TLC showed complete conversion of the starting materials, the solvent was evaporated in vacuum and the residue was used in the next step without any purification [compound X in FIG. 2D]. MS (H⁺): 552.4, ¹H NMR (300 MHz, CDCl₃): δ=7.4 (m, 5H), 4.8 (s, 2H), 4.2 (m, 2H), 2.9 (m, 4H), 2.2 (m, 4H), 1.9-1.5 (m, 8H), 1.5 (s, 9H), 1.4 (s, 9H), 1.1 (m, 4H).

### FIG. 2E

### Protection of α-Amino Group

Compound X (FIG. 2D) was taken in 30 ml DCM. The reaction mixture was stirred for 1 h, cooled and DIEA (0.015 mol) was added, followed by addition of Fmoc chloride (0.006 mol). After stirring over night, DCM (40 ml) was added and the organic phase was washed twice with 1N HCl, brine and dried on Na₂SO₄. After filtration the solvent was evaporated and residue was chromatographed to afford the pure building unit [compound. XI in FIG. 2E]. MS (H⁺): 774.4, ¹H NMR (300 MHz, CDCl₃): δ=7.8 (d, 2H), 7.6 (d, 2H), 7.4 (m, 9H), 4.8 (s, 2H), 4.2 (m, 2H), 2.9 (m, 4H), 2.2 (m, 4H), 1.9-1.5 (m, 8H), 1.5 (s, 9H), 1.4 (s, 9H), 1.1 (m, 4H).

### FIG. 2F

### Removal of BOC Protecting Group

Compound XI (FIG. 2E) was taken in 4N dioxane/HCl. The reaction mixture was allowed to stand overnight at 4° C. The solvent was evaporated in vacuum and the residue [compound XII in FIG. 2F] was used in the next step without any purification. MS (H⁴): 618.3, ¹H NMR (300 MHz, CDCl₃): δ=7.8 (d, 2H), 7.5 (d, 2H), 7.3 (m, 9H), 4.6 (s, 2H), 4.1 (m, 2H), 2.8 (m, 414), 2.2 (m, 4H), 1.9-1.5 (m, 8H), 1.2 (m, 4H).

### FIG. 2G

### Ring Closure

The residue (0.0026 mol) was dissolved in CH₃CN and pyridine (0.0104 mol) was added. After a few minutes DCC (0.0052 mol) was added and the solution was stirred at room temperature. After 3 h the CH₃CN is removed under reduced pressure and the residue extracted with DCM (3×50 ml). The combined organic layers were washed with aqueous HCl (1N), brine (2×50 ml) and dried (MgSO₄). Evaporation of the solvent in a rotary evaporator gave a white powder. The residue was chromatographed to afford the pure scaffold [compound XIII in FIG. 2G]. MS (H⁺): 600.3, ¹H NMR (300 MHz, CDCl₃): δ=7.8 (d, 2H), 7.6 (d, 214), 7.5 (m, 9H), 4.8 (s, 2H), 4.2 (m, 2H), 2.7 (m, 4H), 2.4 (m, 4H), 1.9-1.5 (m, 8H), 1.3 (m, 4H).

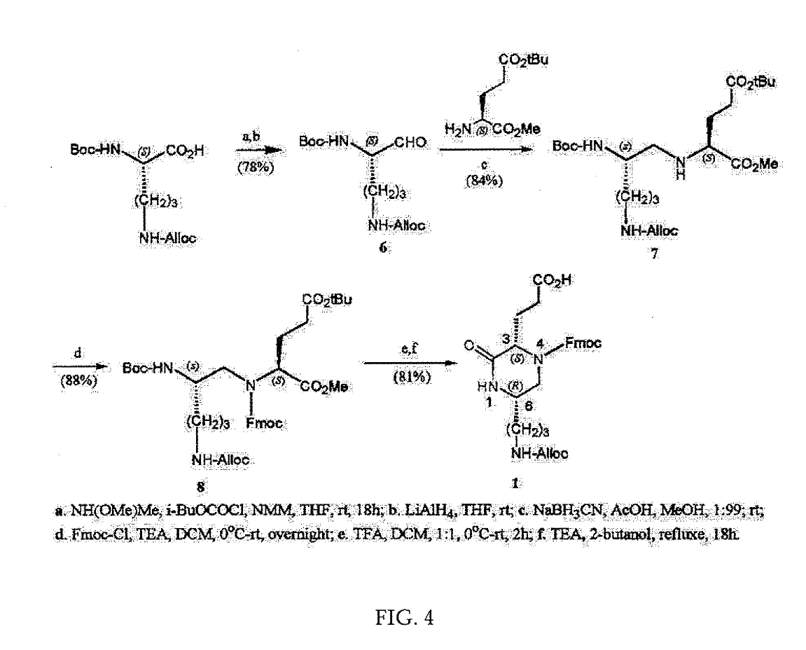

### Preparation of Scaffolds (FIG. 4)

### FIG. 4

### Boc-(S)Orn-(Alloc)-H (Compound 6)

To a solution of commercial Boc-(S)Orn-(Alloc)-OH (3.53 g, 12 mmol) in DCM (50 mL) was added N-methylmorpholine (2.6 mL, 24 mmol). The mixture was cooled to −15° C., and isobutyl chloroformate (1.81 g, 12 mmol) was added. After 15 min at this temperature, N,O-dimethylhydroxylamine hydrochloride (1.02 g, 14 mmol) was added. The mixture was stirred at −15° C. for 1 h, allowed to warm to room temperature, and stirred for additional 3 h. The reaction mixture was poured into H₂O (40 mL), and the aqueous phase was extracted with DCM (2×40 mL). The combined organic extracts were dried over Na₂SO₄ and the solvent was removed in vacuo to give crude hydroxamate which after purification by flash chromatography (50% EtOAc/PE) yielded pure intermediate hydroxamate (3.80 g, 90%) as a colorless oil. R_{f}=0.35 (EtOAc/PE, 1:1), MS m/z 382 (MNa⁺, 100); ¹H NMR 5.91 (m, 1H), 5.36 (dd, J=10, 2 Hz, 1H), 5.20 (dd, J=16, 2 Hz, 1H), 4.48 (d, J=5 Hz, 2H), 4.33 (m, 2H), 3.62 (s, 3H), 2.75 (s, 3H), 2.19 (m, 2H), 1.9-1.5 (m, 2H), 1.42 (s, 9H), 1.10 (m, 2H). To the solution of the hydroxamate (2.57 g, 7 mmol) in THF (100 mL) LiAlH₄ (0.53 g, 14 mmol) was added in several portions and the reaction mixture was stirred under N₂ for 40 min in an ice bath. A solution of KHSO₄ (1.35 g) in 30 mL H₂O was added, and the TI-IF was removed under reduced pressure. The residue was extracted with ether (4×40 mL). The combined organic layers were washed with 1N HCl solution (3×40 mL), sat. NaHCO₃ (40 mL) and brine (40 mL) and then dried (MgSO₄). Evaporation of the solvent gave crude aldehyde 6, FIG. 4 as colorless oil (1.58 g, 88% yield), which was used in the next step without further purification. R_{f}=0.75 (EtOAc/PE, 1:1), MS m/z 323 (MNa⁺, 100); ¹H NMR [symbol] 9.05 (bs, 1H), 5.75 (m, 1H), 5.40 (dd, J=10, 2 Hz, 1H), 5.15 (dd, J=16, 2 Hz, 1H), 4.52 (d, J=5Hz, 2H), 4.15 (m, 2H), 2.27 (m, 2H), 1.9-1.5 (m, 2H), 1.42 (s, 9H), 1.13 (m, 2H).

### FIG. 4

### Boc-(S)Orn-(Alloc)-(S)Glu-(t-Bu)OMe pseudodipeptide diester (Compound 7)

H—(S) Glu(t-Bu)-OMe (1.04 g, 5 mmol) was added to 30 mL of MeOH/AcOH (99:1) until the solution became clear. Then aldehyde 6 (FIG. 4) (1.42 g, 5 mmol) was added in 10 mL of MeOH/AcOH (99:1), the reaction mixture was stirred for 1 h at rt, followed by portion wise addition of NaCNBH₃ (039 g, 6.5 mmol). When TLC showed complete conversion of the starting materials, the solvent was evaporated in vacuo and the residue compound 7 in FIG. 4 was used in the next step without any purification. The yield of conversion was estimated by calculation of the area under a peak by HPLC (138 g, 84% yield), [a]_{D} (CHCl₃): +23; MS m/z 502 (M⁺, 100); ¹H NMR [symbol] 5.72 (m, 1H), 5.33 (dd, J=10, 2 Hz, 1H), 5.22 (dd, J=16, 2 Hz, 1H), 4.33 (d, J=5 Hz, 2H), 4.25 (m, 2H), 3.61 (s, 3H), 2.92 (m, 4H), 2.20 (m, 4H), 1.9-1.5 (m, 8H), 1.50 (s, 9H), 1.46 (s, 9H), 1.15 (m, 2H).

### FIG. 4

### Fmoc protected Boc-(S)Orn(Alloc)-(S)Glu(t-Bu)-OMe pseudodipeptide (Compound 8)

Compound 7 in FIG. 4 (1.32g, 2.65 mmol) was taken in 30 mL DCM. The reaction mixture was stirred for a few minutes, cooled and TEA (1.5 mL, 15 mmol) was added, followed by addition of Fmoc chloride (0.59 g, 2.65 mmol). After stirring overnight, DCM (40 mL) was added and the organic phase was washed sequentially twice with cold 0.5N HCl and brine and dried over Na₂SO₄. After filtration, the solvent was evaporated and the residue was chromatographed (EtOAc/PE, 1:1) to afford 1.82 g (88% yield) of pure compound 8 in FIG. 4. R_{f}=0.70 (EtOAc/PE, 1:2.5), [a]_{D} (CHCl₃): +19; MS m/z 609 (M⁺-Boc, 100); ¹H NMR δ 7.80 (d, J=8 Hz, 2H), 7.63 (d, J=8 Hz, 2H), 7.44 (t, J=8 Hz, 2H), 7.40 (t, J=8 Hz, 2H), 5.75 (m, 1H), 5.39 (dd, J=10, 2 Hz, 1H), 5.22 (dd, J=16, 2 Hz, 1H), 4.50, (m, 2H), 4.45 (d, J=5 Hz, 2H) 4.35 (t, J=6 Hz, 1H), 4.00 (m, 2H), 3.84 (3H, 3H), 2.9 (m, 4H), 2.2 (m, 4H), 1.9-1.5 (m, 8H), 1.55 (s, 9H), 1.42 (s, 9H), 1.1 (m, 2H). ¹³C NMR 179.1, 170.3, 168.5, 166.2, 157.0, 155.4, 143.7, 141.8, 131.8, 127.3, 127.3, 124.2, 118.3, 118.3, 78.9, 77.3, 67.5, 67.2, 65.9, 59.4, 47.3, 44.0, 42.5, 40.3, 33.2, 29.4, 29.0, 28.2, 23.6, 21.9.

### FIG. 4

### Fmoc/Alloc Orthogonally Protected 2-Ketopiperazine Carboxylic Acid (Compound 1)

Compound 8 in FIG. 4 (1.16 g, 1.36 mmol) was carefully dissolved in ice bath cooled trifittoroacetic acid in DCM (1:1, 30 mL). The reaction mixture was left to warm to room temperature and after 2 h the solvent was removed by repeated evaporation with DCM (50 mL, 3 times) in vacuum. To the resulting viscous residue was added 2-butanol (40 mL) and NMM (0.14 mL, 136 mmol). The resulting reaction mixture was refluxed overnight. The solvent was evaporated to afford an oily residue, which after purification gave 0.77 g (81%) of compound 1 in FIG. 4; [a]_{D} (CHCl₃): +30, HRMS m/z 521.2478 (MH⁺, calculated 521.2162 for C₃₀H₃₅N₃O₇); ¹H NMR (DMSO-d₆): δ 7.61 (d, J=8 Hz, 2H), 7.54 (d, J=8 Hz, 2H), 7.36 (t, J=8 Hz, 2H), 7.22 (t, J=8 Hz, 2H), 6.30 (m, 1H), 5.30 (dd, J=12, 10 Hz, 1H), 5.24 (bd, 1H), 4.47 (s, 2H), 4.23 (t, J=6 Hz, 1H), 4.12 (m, 2H), 2.63 (m, 4H), 2.10 (m, 4H), 1.8-1.4 (m, 8H), 1.25 (m, 2H); ¹³C NMR δ 175.7, 168.3, 168.0, 165.2, 154.3, 142.3, 141.4, 131.2, 129.3, 126.3, 125.2, 117.5, 117.0, 67.0, 66.5, 65.1, 46.7, 44.3, 42.7, 33.9, 28.4, 28.0, 24.6, 22.9.

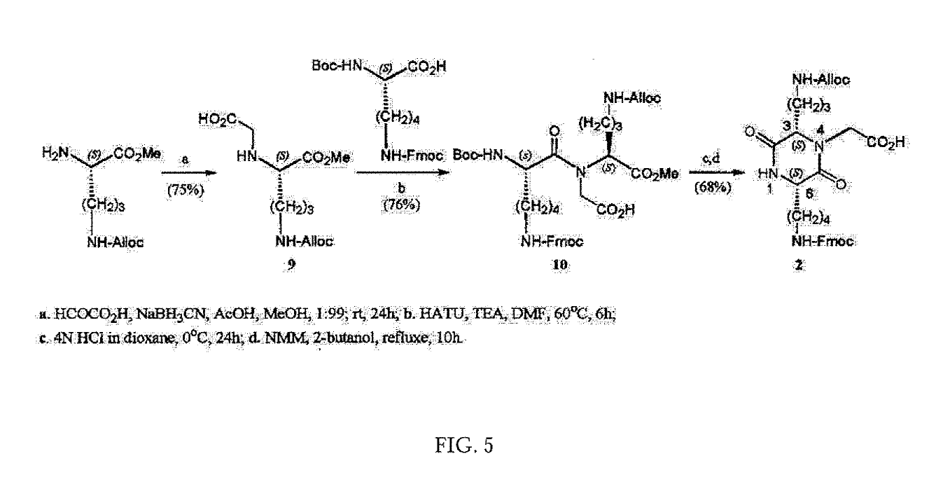

### Preparation of Scaffolds (FIG. 5)

### FIG. 5

### N-(methylenecarboxy)-(S)Orn-(Alloc)-OMe (Compound 9)

Compound 9 in FIG. 5 was prepared from commercial H—(S)Orn-(Alloc)-OMe (2.44 g, 10 mmol) and glyoxylic acid monohydrate (0.92 g, 10 mmol) by the same procedure as for compound 7 in FIG. 4, with a 75% yield (HPLC conversion). The residue was used in the next step without any purification. MS m/z 289 (M⁺, 100); ¹H NMR [symbol] 6.34 (m, 1H), 5.38 (dd, J=12, 10 Hz, 1H), 5.18 (bd, 1H), 4.11 (m, 2H), 3.98 (s, 3H), 2.99 (bs, 2H), 2.35 (m, 2H) 1.8-1.4 (m, 3H), 1.25 (m, 2H).

### FIG. 5

### N^{α}-Boc-(S)Lys-(Fmoc)-N⁽′⁾-(CH₂CO₂H)—(S)Orn-(Alloc)-OMe (Compound 10)

Compound 10 in FIG. 5 was synthesized from compound 9 (FIG. 5) (4.06 g, 14 mmol) and commercial Boc-(S)-Lys-(Fmoc)-OH (6.52 g, 14 mmol) in DMF by heating at 60° C. with HATU (6.35 g, 16.8 mmol) and DIEA (5.7 mL, 50 mmol) for 6 h. After evaporation of the solvent, the residue was taken in DCM (100 mL) and washed twice with 1N citric acid and brine. Purification by flash chromatography (EtOAc) gave pure compound 10 (FIG. 5) (7.15 g, yield 76%) as colorless oil. R_{f}=0.35 (EtOAc) [a]_{D} (CHCl₃): +12; MS m/z 723 (114″, 100); ¹H NMR δ 7.83 (d, J=8 Hz, 2H), 7.63 (d, J=8 Hz, 2H), 7.4 (m, 9H), 6.26 (m, 1H), 5.42 (bd, 1H), 5.27 (bd, 1H), 4.80 (s, 2H), 4.56 (s, 2H), 4.2 (m, 4H), 3.94 (s, 3H), 2.9 (m, 6H), 2.2 (m, 414), 1.9-1.5 (m, 10H), 1.5 (s, 9H), 1.1 (m, 8H).

### FIG. 5

### Fmoc/Alloc Orthogonally Protected 2,5-Diketopiperazine Carboxylic Acid (Compound 2)

Compound 10 (FIG. 5) (1 g, 1.38 mmol) was submitted to Boc removal in 4N HCl dioxane (40 mL) at 0° C. for 24 h, then the solvent and the excess HCl were removed by repeated evaporation with dioxane (3×20 mL). The resulting hydrochloride was cyclized into corresponding diketopiperazine carboxylic acid compound 2 (FIG. 5) in the same manner as for compound 1 (FIG. 4), yielding after chromatography (EtOAc) 0.56 g (68% yield) of colorless oil. R_{f}=0.65 (5% MeOH/EtOAc), [a]_{D} (CHCl₃): +38; HRMS m/z 593.2840 (MH⁺, calculated 593.2413 for C₃₁H₃₆N₄O₈), ¹H NMR (DMSO-d₆): δ 7.54 (d, J=8 Hz, 2H), 7.43 (d, J=8 Hz, 2H), 7.40 (t, J=8 Hz, 2H), 7.20 (t, J=8 Hz, 2H), 5.98 (m, 1H), 5.55 (dd, J=10, 12 Hz, 2H), 5.25 (bd, 2H), 5.11 (s, 2H), 4.40 (bd, 2H), 4.22 (t, J=6 Hz, 1H), 3.88 (bs, 2H), 3.66 (m, 1H), 2.70 (r n, 4H), 1.2-1.5 (m, 10H), ¹³C NMR δ 179.8, 167.1, 165.5, 164.6, 153.2, 143.5, 140.8, 132.0, 125.7, 126.4, 123.5, 119.0, 118.0, 67.0, 64.8, 63.0, 47.0, 46.5, 43.0, 41.9, 40.6, 33.2, 30.2, 27.3, 23.6.

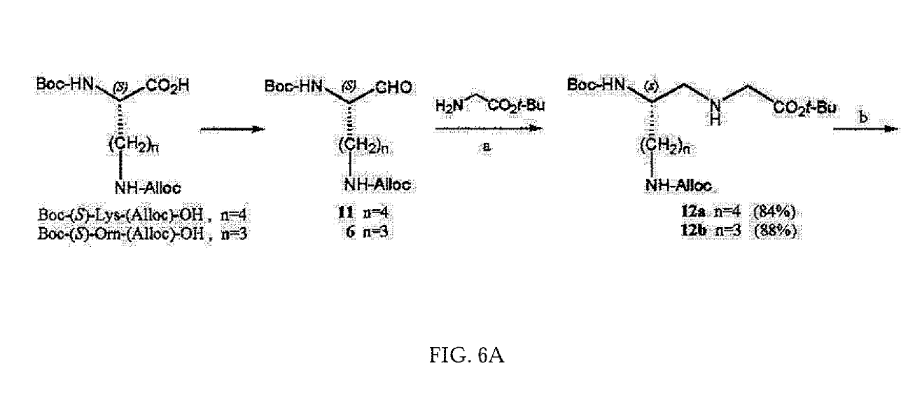

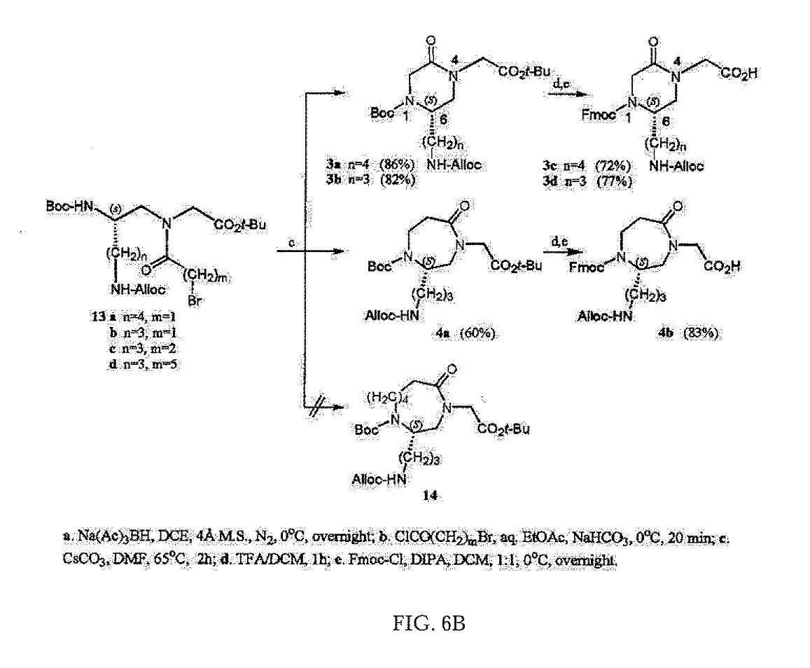

### Preparation of Scaffolds (FIGS. 6 and 6B)

### FIG. 6A

### Boc-(S)Lys-(Alloc)-Gly-O-tBu pseudodipeptide ester (Compound 12a)

Lysinal 11 (FIG. 6A) (1.61 g, 5 mmol) and Gly-O-tBu free base (0.65 g, 5 mmol) were added under N₂ atmosphere to 30 mL, of dry dichloroethane (DCE) in the presence of activated 4 Å molecular sieves and were stirred for 1 h at 0° C. Then NaBH(AcO)₃ (1.42 g, 7 mmol) was added and the reaction mixture was stirred overnight at 0° C. The solvent was evaporated in vacuo and the residue compound 12a (FIG. 6A) was used in the next step without any purification. The yield of conversion was estimated by calculation of the area under a peak by HPLC (1.90 g, 84% yield). [a]_{D} (CHCl₃): +14; MS m/z 430 (M⁺, 50), 330 (M⁺-Boc, 100); ¹H NMR δ 5.70 (m, 1H), 5.37 (dd, J=10, 2 Hz, 1H), 5.23 (dd, J=16, 2 Hz, 1H), 4.33 (d, J=5 Hz, 2H), 4.20 (m, 2H), 2.90 (m, 4H), 2.10 (m, 2H), 1.9-1.5 (m, 4H), 1.51 (s, 9H), 1.48 (s, 9H), 1.15 (m, 2H).

### FIG. 6A

### Boc-(S)Orn-(Alloc)-Gly-O-tBu pseudodipeptide ester (Compound 12b)

Compound 12b (FIG. 6A) was prepared in the same manner as compound 12a (FIG. 6A) from the corresponding ornithinal 6 (FIG. 6A). The yield of conversion was estimated by calculation of the area under a peak by HPLC (2.03 g, 88% yield). [a]_{D} (CHCl₃): +16; MS m/z 416 (M⁺, 60), 316 (M⁺-Boc, 100); ¹H NMR δ 5.70 (m, 1H), 5.37 (dd, J=10, 2 Hz, 1H), 5.23 (dd, J=16, 2 Hz, 1H), 4.33 (d, J=5 Hz, 2H), 4.20 (m, 2H), 2.90 (m, 4H), 2.10 (m, 2H), 1.9-1.5 (m, 4H), 1.51 (s, 9H), 1.48 (s, 9H), 1.15 (m, 2H).

### FIG. 6B

### Boc-(S)Lys-(Alloc)-(NHCOCH₂Br)-Gly-O-tBu pseudodipeptide ester (Compound 13a)

A solution of 2-bromoacetyl bromide (1.2 g, 6 mmol) in EtOAc (10 mL) was added dropwise at 0° C. to a stirring mixture of compound 12a (FIG. 6A) (2.15, 6 mmol) in EtOAc and 1N NaHCO₃ (4:1, 50 mL). After 2 h at 0° C., the mixture was diluted with EtOAc (50 mL) and saturated solution of NaHCO₃ (50 mL) was added. The organic phase was collected and washed with H₂O (20 mL) and brine (20 mL), dried over Na₂SO₄ and concentrated. The crude product was filtered over a silica gel column to yield compound 13a (FIG. 6B), which was used without further purification. R_{f}=0.60 (EtOAc). MS m/z 551, 553 (1:1) (M⁺, 60), ¹H NMR [symbol] 5.90 (m, 1H), 5.46 (dd, J=10, 2 Hz, 1H), 5.34 (dd, J=16, 2 Hz, 1H), 4.45 (s, 2H), 4.26 (d, J=5 Hz, 2H), 4.10 (m, 2H), 2.85 (m, 4H), 2.20 (m, 4H), 1.9-1.5 (m, 6H), 1.45 (s, 9H), 1.40 (s, 9H).

### FIG. 6B

### Boc-(S)Orn-(Alloc)-(NHCOCH₂Br)Gly-O-tBu pseudodipeptide ester (Compound 13b)

Compound 13b (FIG. 6B) was prepared in the same manner as compound 13a (FIG. 6A) from corresponding compound 12b (FIG. 6A) and 2-bromoacetyl bromide. R_{f}=0.60 (EtOAc). MS m/z 535, 537 (1:1) (M⁺, 100), ¹H NMR [symbol] 5.90 (m, 1H), 5.45 (dd, J=10, 2 Hz, 1H), 5.35 (dd, J=16, 2 Hz, 1H), 4.50 (s, 2H), 4.23 (d, J=5 Hz, 2H), 4.10 (m, 2H), 2.70 (m, 4H), 2.15 (m, 4H), 1.9-1.5 (m, 4H), 1.47 (s, 9H), 1.40 (s, 9H).

### FIG. 6B

### Boc-(S) Orn-(Alloc)-(NHCOCH₂CH₂Br)-Gly-O-tBu pseudodipeptide ester (Compound 13c)

Compound 13c (FIG. 6B) was prepared in the same manner as compound 13a (FIG. 6A) from corresponding compound 12b (FIG. 6A) and 3-bromo propionyl chloride. R_{f}=0.60 (EtOAc). MS m/z 551, 553 (1:1) (M⁺, 80), ¹H NMR [symbol] 5.90 (m, 1H), 5.45 (dd, J=10, 2 Hz, 1H), 5.35 (dd, J=16, 2 Hz, 1H), 4.23 (d, J=5 Hz, 2H), 4.10 (m, 2H), 3.60 (m, 2H), 2.65 (m, 4H), 2.20 (m, 6H), 1.9-1.5 (m, 4H), 1.45 (s, 9H), 1.42 (s, 9H).

### FIG. 6B

### Boc-(S) Orn-(Alloc)-(NHCO(CH₂)₅Br)-Gly-O-tBu pseudodipeptide ester (Compound 13d)

Compound 13d (FIG. 6B) was prepared in the same manner as compound 13a (FIG. 6A) from the corresponding compound 12b (FIG. 6A) and 6-bromo hexanoyl chloride. R_{f}=0.60 (EtOAc). MS m/z 614, 616 (1:1) (MNa⁺, 40), 592, 594 (1:1) (M⁺, 60), ¹H NMR δ 5.95 (m, 1H), 5.45 (dd, J=10, 2 Hz, 1H), 5.35 (dd, J=16, 2 Hz, 1H), 4.32 (d, J=5 Hz, 2H), 4.10 (m, 2H), 3.50 (m, 2H), 2.60 (m, 4H), 2.20 (m, 6H), 1.9-1.5 (m, 10H), 1.50 (s, 9H), 1.40 (s, 9H).

### FIG. 6B

### General Procedure for Ring Closure of Compounds 13a-c to Compounds 3a, b and 4a

Compounds 13a, 13b or 13c (FIG. 68) (5 mmol) was dissolved in 30 mL of dry DMF. Cs₂CO₃ (10 mmol) was added and the reaction mixture was heated to 65° C. under N₂ atmosphere with vigorous stirring. After 2 h at this temperature, the solvent was evaporated, the residue was taken into DCM (100 mL) and washed twice with 1N citric aid (50 mL), brine (50 mL) and dried over Na₂SO₄. After filtration, the solvent was evaporated and the oily residue was chromatographed (EtOAc/PE, 1:1) to give the desired product.

Compound 3a (FIG. 6B): 0.67 g colorless oil (86% yield), R_{f}=0.8 (EtOAc/PE 1:1), [a]_{D} (CHCl₃): +23; MS m/z 470 (M⁺, 20), 370.3 (M⁺-Boc, 20), ¹H NMR δ 5.92 (m, 1H), 5.38 (dd, J=12, 10 Hz, 2H), 5.20 (dd, J=12, 10 Hz, 2H), 5.11 (bs, 2H), 4.40 (bd, 2H), 3.88 (bs, 2H), 3.66 (m, 1H), 2.70 (m, 2H), 1.8-1.2 (m, 24H).
Compound 3b (FIG. 6B): 0.59 g colorless oil (82% yield), R_{f}=0.8 (EtOAc/PE 1:1), [a]_{D} (CHCl₃): +25; MS m/z 456 (M⁺, 20), 356 (M⁺-Boc, 80), ¹H NMR [symbol] 5.98 (m, 1H), 5.36 (bd, 2H), 5.22 (bd, 2H), 5.10 (bs, 2H), 4.45 (bd, 2H), 3.80 (bs, 2H), 3.70 (m, 1H), 2.60 (m, 2H), 1.8-1.2 (m, 22H).
Compound 4a (FIG. 6B): 0.42 g colorless oil (60% yield), R_{f}=0.8 (EtOAc/PE, 1:1), [a]_{D} (CHCl₃): +14; MS m/z 492 (MNa⁺, 90), 470 (M⁺, 50), 370 (M⁺-Boc, 80); ¹H NMR [symbol] 5.95 (m, 1H), 5.40 (dd, J=12, 10 Hz, 2H), 5.20 (bd, 2H), 5.11 (bs, 2H), 4.40 (bd, 2H), 3.88 (bs, 2H), 3.66 (m, 1H), 2.70 (m, 2H), 2.15 (m, 28), 1.8-1.2 (m, 22H).

### FIG. 6B

### General Procedure for the Synthesis of Compounds 3c, d and 4b

Compounds 3a, 3b or 4a (FIG. 6B) (1.36 mmol) was carefully dissolved in ice bath cooled trifluoroacetic acid in DCM (1:1, 30 mL). The reaction mixture was left to warm to room temperature and after 2 h the solvent was removed by repeated evaporation with DCM (50 mL, 3 times) in vacuum giving viscous reddish oil, which was used in the next step without further purification. In the next step the reddish oil was taken into 50 mL of DCM and 10 mmol of DIEA was added at 0° C. Fmoc-Cl (1.2 mmol) was added in small portions and the reaction mixture was left overnight at rt. Then, additional 50 mL DCM was added and the organic phase was washed twice with 1N citric acid, sat. NaHCO₃ and brine. After drying over Na₂SO₄ and subsequent filtration, the solvent was evaporated and the final crude products compounds 3c, d and 4b (FIG. 6B) was purified by flash chromatography.

### FIG. 6B

### (S)-2-(5-(allyloxycarbonylamino)butyl)-4-(2-fluorenyl-2-oxoethyl)-5-oxopiperazine-1-carboxylic acid (Compound 3c)

0.65 g colorless oil (72% yield), R_{f}=0.35 (10% MeOH/EtOAc), [a]_{D} (CHCl₃): +17; FIRMS m/z 536.2780 (MH⁺, calculated 536.2319 for C₂₉H₃₃N₃O₇), ¹H NMR [symbol] 7.80 (d, J=8 Hz, 2H), 7.60 (bd, 2H), 7.42 (t, J=8 Hz, 2H), 7.30 (bt, 2H), 5.95 (m, 1H), 5.38 (bd, 2H), 5.20 (bd, 2H), 5.00 (s, 2H), 4.60-4.20 (m, 10H), 3.88 (bs, 2H), 3.66 (m, 3H), 3.18 (m, 2H), 1.5-1.2 (m, 8H), ¹³C NMR 174.0, 165.3, 160.8, 162.1, 142.5, 141.2, 131.0, 128.2, 125.0, 120.5, 120.0, 117.0, 66.0, 65.1, 64.2, 48.7, 46.9, 42.5, 42.0, 41.0, 30.4, 29.9, 20.5.

### FIG. 6B

### (S)-2-(5-(allyloxycarbonylamino)propyl)-4-(2-fluorenyl-2-oxoethyl)-5-oxopiperazine-1-carboxylic acid (compound 3d)

0.68 g colorless oil (77% yield), R_{f}=0.35 (10% MeOH/EtOAc), [a]_{D} (CHCl₃): +18; HRMS m/z 521.2452 (MH⁺, calculated 521.2162 for C₂₈H₃₁N₃O₇), ¹H NMR [symbol] 7.70 (bd, 2H), 7.55 (bd, 2H), 7.40 (bt, 2H), 7.28 (bt, 2H), 5.90 (m, 1H), 5.30 (bd, 2H), 5.23 (bd, 2H), 5.05 (s, 2H), 4.60-4.20 (m, 10H), 3.82 (bs, 2H), 3.54 (m, 3H), 3.10 (m, 2H), 1.5-1.2 (m, 6H), ¹³C NMR δ 175.1, 163.3, 162.8, 161.1, 143.2, 140.2, 130.2, 127.8, 124.0, 122.3, 121.2, 118.6, 65.0, 64.5, 63.1, 45.2, 45.0, 44.7, 43.6, 42.0, 28.5, 22.7.

### FIG. 6B

### (S)-2-(5-(allyloxycarbonylamino)propyl)-4-(2-fluorenyl-2-oxoethyl)-5-oxodiazepane-1-carboxylic acid (Compound 4b)

0.71 g colorless oil (83% yield), R_{f}=0.40 (3% MeOH/AtOAc), [a]_{D} (CHCl₃): +12; FIRMS m/z 536.2643 (MH⁺, calculated 536.2319 for C₂₉H₃₃N₃O₇), ¹H NMR [symbol] 7.78 (d, J=8 Hz, 2H), 7.57 (d, J=8 Hz, 2H), 7.35 (t, J=8 Hz, 2H), 7.26 (t, J=8 Hz, 2H), 5.90 (m, 1H), 5.60 (bs, 1H), 5.33 (bd, 2H), 5.20 (bd, 2H), 4.56 (bd, 2H), 4.40 (m, 1H), 4.18 (m, 2H), 3.90 (bs, 1H), 3.66 (m, 1H), 3.18 (m, 2H), 1.5-1.2 (m, 81-1), ¹³C NMR [symbol] 175.4, 163.0, 162.2, 160.5, 141.4, 140.7, 133.3, 126.4, 124.0, 122.2, 121.1, 118.4, 66.0, 65.5, 63.0, 62.3, 45.8, 45.2, 43.7, 41.3, 40.9, 31.6, 19.8.

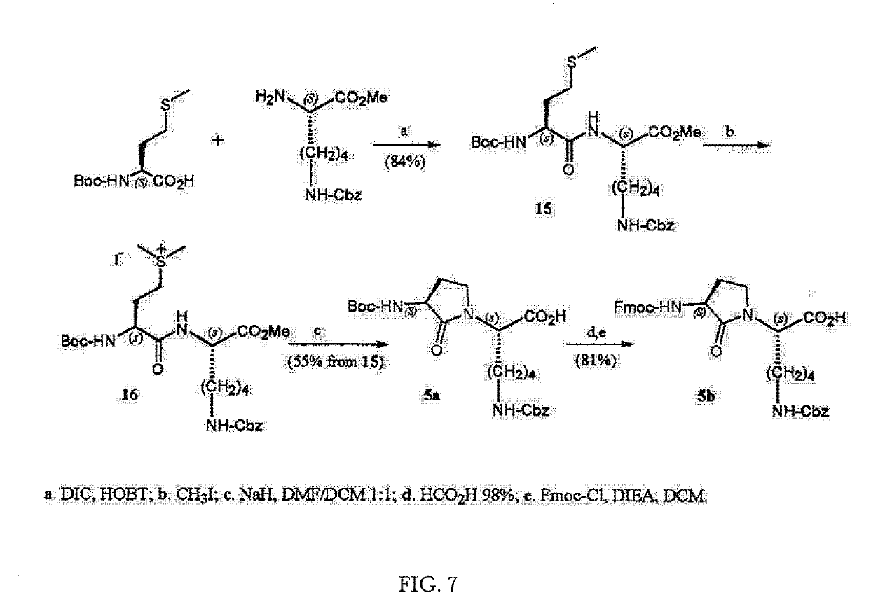

### Preparation of Scaffolds (FIG. 7)

### FIG. 7

### Boc-(S) Met-(S) Lys-(Cbz)-OMe (Compound 15)

(S) Lysine-(Cbz)-methyl ester hydrochloride (1 g, 3 mmol), HOBT (0.4 g, 3 mmol), t-butyloxycarbonyl-(S)-Methionine (0.8 g, 3 mmol) and N,N-Diisopropylethylamine (1.05 mL, 6 mmol) were dissolved in dry THF (15 mL), the solution was cooled in an ice-water bath and diisopropylcarbodiimide (0.4 g, 3.15 mmol) was added. Stirring was continued for 1 h at 0° C. and an additional hour at rt. The solvent was evaporated in vacuo. A mixture of EtOAc (15 mL) and sat. NaHCO₃ (7.5 mL) was added to the residue and the organic phase was sequentially extracted with 10% citric acid in water, sat. NaHCO₃ and water (7.5 mL each). The solution was dried over anhydrous Na₂SO₄, filtered and evaporated to dryness. The residue was triturated with hexane, filtered, washed with hexane and dried. The crude dipeptide derivative compound 15 (FIG. 7) (1.80 g,) was purified by chromatography on basic alumina (EtOAc) (1.46 g, 84% yield) ¹H NMR δ 8.89 (bd, 1H), 7.85 (s, 5H), 5.11 (s, 2H), 3.71 (s, 3H), 3.2 (m, 2H), 2.5 (t, J=6 Hz, 2H), 2.1-2.3 (m, 5H), 2.1-1.1 (m, 8H), 1.42 (s, 9H).

### FIG. 7

### Boc-Met-Lys-(Cbz)-Methyl Ester Methylsulfonium Iodide (Compound 16)

Boc-Met-Lys(Cbz)-OMe (compound 15, FIG. 7, 18.7 g) was dissolved in CH₃I (60 mL) and stirred at rt for 3 days. Concentration in vacuo gave an amorphous solid (19.1 gr, 95% yield): ¹H NMR [symbol] 8.89 (d, J=7 Hz, 1H), 7.8 (s, 5H), 6.03 (d, J=7 Hz, 1H), 5.37 (m, 1H), 5.11 (s, 2H), 4.7-4.3 (m, 2H), 3.71 (s, 3H), 3.3-3.0 (m, 2H), 3.25 (m, 3H) 3.2 (m, 2H), 3.1 (s, 3H), 2.1-1.1 (m, 8H), 1.42 (s, 9H).

### FIG. 7

### (S)3-[(t-Butoxycarbonyl)amino]-2-oxo-lpyrralidine-(S)-6-[(benzyloxycarbonyl)amino]-2-heptanoic Acid (Compound 5a)

The sulfonium salt 16 (FIG. 7, 10 g, 15.3 mmol) was dissolved in 300 mL of 1:1 DMF-CH₂Cl₂ under N₂ and cooled to 0° C. NaH (1.5 g of a 50% mineral oil suspension, 31.5 mmol) was added at once, and the mixture was stirred at 0° C. for 2.5 h. Ethyl acetate (100 mL) followed by water (24 mL) was added, and the resultant solution was left overnight at rt. The solution was concentrated in vacuo to a small volume and partitioned between water (50 mL) and CH₂Cl₂ (50 mL). The phases were separated, and the aqueous phase was acidified to pH 4 with 0.5 M citric acid. Continuous extraction with CH₂Cl₂ followed by concentration in vacuo gave crystalline product compound 5a (FIG. 7, 4.6 g, 58% yield): mp 137-139° C. Recrystallization from EtOAc gave 4 gr of the product: mp 141.5-143° C.; [a]_{D} (CHCl₃): +21; MS m/z 477 (M⁺-Boc, 100); ¹H NMR δ 7.8 (s, 5H), 5.11 (s, 2H), 4.54 (dd, J=11, 6 Hz, 1H), 4.3 (br t, J=9 Hz, 1H), 3.5-3.2 (m, 2H), 3.1 (t, J=6 Hz, 2H), 2.1-21.1 (m, 8H), 1.42 (s, 9H).

### FIG. 7

### (S)2-((S)-3-(((9H-fluoren-9-yl)methoxy)carbonylamino)-2-oxopyrrolidin-1-yl)-6-(benzyloxycarbonylamino)-2-methylhexanoic acid (Compound 5b)

Compound 5a (FIG. 7, 4 gr, 8.2 mmol) was dissolved in 98% HCO₂H (50 mL) and the solution was kept at rt for 2 hr. After removal of the excess formic acid in vacuo, the residue was dissolved in 50 mL of DCM at 0° C., then diisopropylethylamine (8.5 g=10:2 mL, 65.6 mmol) and Fmoc chloride (2.12 gr=8.2 mmol) were added, and the resultant solution was left overnight with vigorous stirring. The organic layer was extracted with 1N HCl, and then twice with brine. The solution was dried over anhydrous Na₂SO₄ filtered and evaporated to dryness in vacuo. Recrystallization from EtOAc afforded 5.4 gr of compound 5b (FIG. 7) (81% yield). [a]_{D} (CHCl₃): +24; HRMS m/z 586.2530 (MH⁺, calculated 586.2475 for C₃₃H₃₅N₃O₇), ¹H NMR (DMSO-d₆) δ 7.8-7.2 (s, 13H); 5.11 (s, 2H), 4.54 (dd, J=11, 6 Hz, 1H), 4.3 (br t, J=9 Hz, 1H), 3.5-3.2 (m, 2H), 3.1 (t, J=6 Hz, 2H), 2.1-1.1 (m, 8H), ¹³C NMR [symbol] 176.2, 171.0, 153.9, 152.3, 138.2, 130.3, 127.4, 126.8, 126.0, 118.0, 117.6, 67.0, 66.5, 65.4, 44.2, 43.1, 41.1, 34.5, 28.9, 27.2, 21.5.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the invention.

Section headings are used herein to ease understanding of the specification and should not be construed as necessarily limiting.

1-14. (canceled)

15. An orthogonally-protected heterocyclic chiral scaffold comprising a non-aromatic heterocyclic moiety of between 6 and 9 atoms, said heterocyclic moiety including:
a first nitrogen atom;
a first mono substituted carbon atom substituted with a —CH₂—R1 moiety, R1 selected from the group consisting of (CH₂)k-COOH and (CH₂)k-Q1-P1, wherein said first monosubstituted carbon atom constitutes a first chiral center of said heterocyclic moiety;
a second mono substituted carbon atom substituted with a —CH₂—R2 moiety, R2 selected from the group consisting of (CH₂)m-COOH and (CH₂)m-Q2-P2, wherein said second monosubstituted carbon atom constitutes a second chiral center of said heterocyclic moiety;
a second nitrogen atom substituted with an —R3 moiety, not ortho to said first nitrogen atom, R3 selected from the group consisting of P3, (CH₂)o-COOH and (CH₂)o-P3;
the remaining 2 to 5 atoms of said heterocyclic moiety being independently selected from the group consisting of CH₂ and CO,
and
wherein k, m and o are integers independently between 0 and 5;
wherein Q1 and Q2 are independently selected from the group consisting of NH, O and S;
wherein P1, P2 and P3 are protecting groups for a respective Q1, Q2 and said second nitrogen atom, each one of P1, P2 and P3 present being independently selected; and
wherein R3 is selected from the group consisting of (CH₂)o-COOH and (CH₂)o-P3, so that the scaffold comprises three side chains around said heterocyclic moeity.

16. The scaffold of claim 15, wherein each one of P1, P2, and P3 present are independently:
selected from the groups consisting of Alloc, Fmoc, TFA, CBZ, Boc, o-Nosyl, Mtt, Ddz Dde, Bpoc, NVOC, NBoc and Teoc when bonded to an NH moiety;
selected from the group consisting of Alloc, Allyl, Bz, Dmb, Fmoc, Pivaloyl and Ac when bonded to an O atom; and
selected from the group consisting of Acm, Trt and StBu when bonded to an O atom.

17. The scaffold of claim 15, wherein said heterocyclic moiety is selected from the group consisting of piperazines, ketopiperazines and diketopiperazines.

18. The scaffold of claim 15, having a structure of the formula: 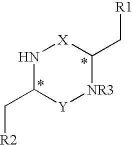
wherein:
X and Y are independently selected from the group consisting of CO and CH₂;
R1 is selected from the group consisting of (CH₂)k-COOH and (CH₂)k-Q1-P1;
R2 is selected from the group consisting of (CH₂)m-COON and (CH₂)m-Q2-P2; and
R3 is selected from the group consisting of P3, (CH₂)o-COOH and (CH₂)o-P3.

19. A scaffold of claim 15, attached to a support useful for solid-phase organic synthesis.

20. A library of compounds, comprising at least two different compounds including a scaffold of claim 15.

21. The library of compounds of claim 20, comprising a plurality of different combinatorially-varying compounds including a said scaffold.

22. An orthogonally-protected heterocyclic chiral scaffold comprising: a non-aromatic heterocyclic moiety of between 6 and 9 atoms, said heterocyclic moiety including
a first nitrogen atom;
a first mono substituted carbon atom substituted with a —CH₂—R1 moiety, R1 selected from the group consisting of (CH₂)k-COOH and (CH₂)k-Q1-P1, wherein said first monosubstituted carbon atom constitutes a first chiral center of said heterocyclic moiety;
a second mono substituted carbon atom substituted with a —CH₂—R2 moiety, R2 selected from the group consisting of (CH₂)m-COOH and (CH₂)m-Q2-P2, wherein said second monosubstituted carbon atom constitutes a second chiral center of said heterocyclic moiety;
a second nitrogen atom substituted with an —R3 moiety, not ortho to said first nitrogen atom, R3 selected from the group consisting of P3, (CH₂)o-COOH and (CH₂)o-P3;
the remaining 2 to 5 atoms of said heterocyclic moiety being independently selected from the group consisting of CH₂ and CO,
and
wherein k, m and o are integers independently between 0 and 5;
wherein Q1 and Q2 are independently selected from the group consisting of NH, O and S;
wherein P1, P2 and P3 are protecting groups for a respective Q1, Q2 and said second nitrogen atom,
each one of P1, P2 and P3 present being independently selected; and
wherein R3 is selected from the group consisting of P3 and (CH₂)o-P3, so that the scaffold comprises two protected side chains R1 and R2 as well as a protected ring nitrogen.

23. The scaffold of claim 22, wherein each one of P1, P2, and P3 present are independently:
selected from the groups consisting of Alloc, Fmoc, TFA, CBZ, Boc, o-Nosyl, Mtt, Ddz Dde, Bpoc, NVOC, NBoc and Teoc when bonded to an NH moiety;
selected from the group consisting of Alloc, Allyl, Bz, Dmb, Fmoc, Pivaloyl and Ac when bonded to an O atom; and
selected from the group consisting of Acm, Trt and StBu when bonded to an O atom.

24. The scaffold of claim 22, wherein said heterocyclic moiety is selected from the group consisting of piperazines, ketopiperazines and diketopiperazines.

25. The scaffold of claim 22, having a structure of the formula: 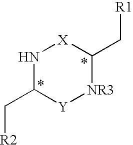
wherein:
X and Y are independently selected from the group consisting of CO and CH₂;
R1 is selected from the group consisting of (CH₂)k-COOH and (CH₂)k-Q1-P1;
R2 is selected from the group consisting of (CH₂)m-COOH and (CH₂)m-Q2-P2; and
R3 is selected from the group consisting of P3, (CH₂)o-COOH and (CH₂)o-P3.

26. A scaffold of claim 22, attached to a support useful for solid-phase organic synthesis.

27. A library of compounds, comprising at least two different compounds including a scaffold according to claim 22.

28. The library of compounds of claim 27, comprising a plurality of different combinatorially-varying compounds including a said scaffold.
